(19) United States
(12) Patent Application Publication
(10) Pub. No.: US 2003/0054047 A1
(43) Pub. Date: Mar. 20, 2003

(54) Pharmaceutical composition for the treatment of viral infection

(75) Inventor: Xinxian Zhao, Shenzhen (CN)

Correspondence Address: Albert Wai-Kit Chan, Law Offices of Albert Wai-Kit Chan, LLC, World Plaza, Suite 604, 141-07 20th Avenue, Whitestone, NY, 11357, US

(73) Assignee: Sanjiu Medical & Pharmaceutical Co., Ltd.

(21) Appl. No.: 10/172,319

(22) Filed: Jun. 13, 2002

Related U.S. Application Data
(60) Provisional application No. 60/298,077, filed on Jun. 15, 2001.

(51) Int. Cl. A61K 35/78

(52) U.S. Cl. 424/725

(57) ABSTRACT

This invention provides a method of treating viral infection with an herbal composition comprising an active ingredient from herbs of at least one species selected from genus Isatis and the genus Baphicacanthus; formulating the extract into a pharmaceutically acceptable formulation; and administering said formulation to a mammal suffering viral infection. This invention also provides for a method of treating viral infection with an herbal composition wherein the formulation is in the form of a powder, syrup, tea, tincture, injection, topical solution, capsule, pill, granule, tablet, nebula, suppository or microcapsule.

This application claims priority of U.S. Serial No. 60/298,077, filed Jun. 15, 2001, the content of which is incorporated by reference here into this application.

Throughout this application, various references are referred to. Disclosures of these publications in their entireties are hereby incorporated by reference into this application to more fully describe the state of the art to which this invention pertains.

### BACKGROUND OF THE INVENTION

1. Field of the Invention

The present invention relates to the field of herbal pharmaceutical formulations. In particular, the present herbal pharmaceutical composition is related to the treatment of the influenza virus through administration of extracted herbs from the genus Isatis and the genus Baphicacanthus.

2. Description of the Prior Art

Naturally derived active compositions are commonly used in the prevention and treatment of an ever increasing number of diseases and maladies. With the interest in naturally derived active compounds, the world is looking towards traditional Eastern remedies. Of primary importance in this field is China and its wealth of herbally derived compositions. Many of these remedies have been shown to be as effective as traditional Western, synthetically produced pharmaceuticals but usually lack the detrimental side effects commonly experienced with synthetic products. One illness that has traditionally been treated by a large number of natural remedies is the flu, brought about by infection with the influenza virus.

Influenza is a virus of the respiratory tract, and is an etiological agent of acute bronchitis, pneumonia, croup, and influenza. The influenza virus caused one of history's worst epidemics during the early twentieth century. Influenza is a large RNA virus having a core of helical symmetry containing a soluble nucleoprotein antigen. The virion has a membrane envelope with spikes containing two viral glycoproteins, one having hemagglutinating activity (HA) and one having neuraminidase activity. The influenza virus attaches to a specific glycoprotein receptor for the hemagluttinin on the cell surface. The virus possesses an unusual geometric structure of RNA segments, which reshuffle upon each cycle of infection. This shuffling happens very quickly, making it extremely difficult to treat. Due to such rapid changes, the human immune system finds it difficult to counteract. Current therapy includes use of medication Amantadine, which has various undesirable side effect including anorexia, naupathia, headache, vertigo, insomnia, and ataxia.

The common cold and influenza are major causes of illness an loss of productivity throughout the world. The National Center for Health Statistics estimates that in 1992, 62 million cases of the common cold in the United State required medical attention, and colds caused 157 million days of restricted activity and 15 million days of lost work. Approximately between 75-80 percent of adult colds are caused by viruses including the influenza A and B viruses. Not only are viral induced cold symptoms a major inconvenience to the human population, but are also responsible for huge economic losses to the world as a whole.

Attempts have been made to incorporate natural herbal compounds in the treatment of influenza and its symptoms. For example, U.S. Pat. No. 4,886,666, entitled “Pharmaceutical Composition For Inhibiting Viruses And Increasing Immune Function” discloses a pharmaceutical composition of four active ingredients: 1) Polysaccharide of Wang Qi extracted from Astragalus membranaceus Bge or other species of Astragalus; 2) Banlankesu extracted form among Isatis tinctoria L, Isatis indigotica Fort or Baphicacanthus cusia Bremek; 3) Yejuhua-flavonoid extracted from Crysanthemum indicum L; and 4) Guanahonhsu extracted from among Dryopteris crassirhizoma Nakai, Osmunda japonica Thunb, Lunathyrium acrostichoides ching or Matteuccia stuthiopteris Todaro. The composition is used for the treatment and prevention of infections caused by viruses and increases immune function. Although such a formulation may help in the treatment of infections caused by viruses, it requires the use of multiple ingredients to achieve this effect. Multiple ingredients in a formulation that is administered to mammals suffers from several drawbacks. Primarily, if the function of each ingredient is not understood, it is impossible to determine their individual effects. Not only may combinations of ingredients work less efficiently together, but the activity of some may not be indicated for a particular patient. Many users of such formulations may be suffering from other conditions and administering an ingredient of a composition that functions, for example, in the boosting of immune function might have detrimental effects.

Natural herbal compounds have also been used to treat other viral diseases including hepatitis and the Human Immunodeficiency Virus (HIV). Hepatitis is a disease of the human liver. It is manifested with inflammation of the liver and is usually caused by viral infections and sometimes from toxic agents. Hepatitis may progress to liver cirrhosis, liver cancer, and eventually death. Several viruses such as hepatitis A, B, C, D, E and G are known to cause various types of viral hepatitis. Among them, HBV and HCV are the most serious. HBV is a DNA virus with a virion size of 42 nm. HCV is a RNA virus with a virion size of 30-60 nm. Medications currently in use include Interferon, Acyclovir (ACV), and Interleukin. Side effects of Interferon are fever, chill, muscle ache, headache, hematopoiesis function disorder, and possibly IFN antibody. Acyclovir use also suffers the drawback of side effects such as kidney toxicity and nerve system disorder. Interleukin also may result in fever, chills, and low blood pressure.

The Human Immunodeficiency Virus, and its resultant disease, acquired immunodeficiency syndrome (AIDS) was first reported in 1981, with its causative agent, HIV, discovered in 1983. AIDS has now become a worldwide epidemic with 13 million humans infected worldwide in 1993, 21 million in 1996 and the numbers appear to be escalating at an alarming rate. The most common medication used to treat AIDS patients is Azidovudine, commonly know as AZT. Other medications also exist and are usually combined with AZT to produce drug cocktails. Not only do these medications cause excessive bleeding disorders, liver toxicity and medulla function strain, but their high cost prohibits the inhabitants of developing countries their access.

U.S. Pat. No. 5,178,865, entitled “Chinese Herbal Extracts In the Treatment Of HIV Related Disease In Vitro,” discloses the use of a variety of herbs for inhibiting in vitro HIV infection in T lymphocyte cells and mononuclear phagocytic lineage cells infected with HIV. One of the herbs proposed consists of an extract from Isatis tictoria which was tested for anti-HIV activity though standard laboratory method for T cell toxicity testing. All of the herbs proposed were obtained from China in extract form, packaged in ampoules for parenteral use but may be extracted according to organic extraction procedures.

Although there have been attempts to produce natural herbal products for the treatment of viral diseases, these have been largely unsuccessful due to improper extraction of the active ingredients from their natural source. It is therefore an object of the present invention to provide herbal remedies for the treatment of the influenza virus, HIV, hepatitis and other viral infections. It is a further object of the present invention to provide for the treatment of viral infections without the common side effects experienced when synthetically produced pharmaceuticals are utilized.

It is still another object of the present invention to accomplish the above-stated objects by utilizing an improved method for the extraction of natural herbal products which are more effective in the treatment of viral infections.

The foregoing objects and advantages of the invention are illustrative of those that can be achieved by the present invention and are not intended to be exhaustive or limiting of the possible advantages which can be realized. Thus, these and other objects and advantages of the invention will be apparent from the description herein or can be learned from practicing the invention, both as embodied herein or as modified in view of any variation which may be apparent to those skilled in the art. Accordingly, the present invention resides in the novel methods, arrangements, combinations and improvements herein shown and described.

### SUMMARY OF THE INVENTION

In accordance with these and other objects of the invention, a brief summary of the present invention is presented. Some simplifications and omission may be made in the following summary, which is intended to highlight and introduce some aspects of the present invention, but not to limit its scope. Detailed descriptions of a preferred exemplary embodiment adequate to allow those of ordinary skill in the art to make and use the invention concepts will follow in later sections.

According to a broad aspect of the invention, a method is disclosed for method of treating viral infection with an herbal composition. The method comprises the extraction of an active ingredient from herbs of at least one species selected from the genus Isatis and the genus Baphicacanthus.

According to another broad aspect of the invention, the extract is then formulated into a pharmaceutically acceptable formulation and administered to a mammal suffering viral infection.

### DETAILED DESCRIPTION OF THE FIGURES

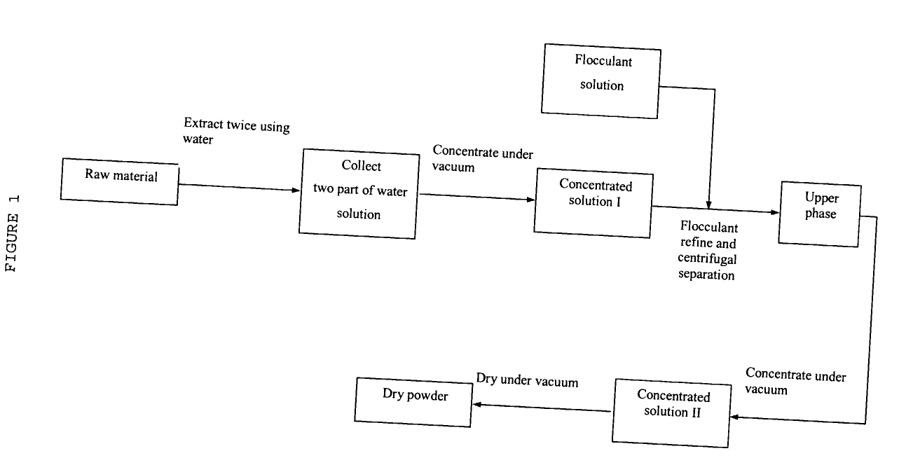

FIG. 1. Extraction Procedures:

1. Weigh certain amount of Isatis indigotica Fort., add 8 times volume of water (V/W) into the weighted herb, then decoct it for two hours, the solution I was obtained after the filtration;

2. The solid residue of the filtration was decocted with 6 times of water (V/W) for 1 hour, the solution II was obtained after the same filtration;

3. Combine the solution I and II, and concentrate it under vacuum condition until the specific density located from 1.01 to 1.10;

4. Preparation of the flocculant solution (1˜5% W/W): weight certain amount of Chitosan, add water to the set volume and appropriate volume of ice acetic acid, keep the temperature be 60C around in a water batch and stir the solution intermittently, till the solution be transparent and with no suspension material.

5. Add flocculant solution (20˜80% volume of the concentrated solution), at the same time stir the solution, after two hours placement, the suspension solution was separated using a centrifugal machine, the clear upper solution was obtained and the precipitate deposited.

6. Concentrate the clear upper solution under vacuum condition and dry the concentrated solution at 60° C. in a vacuum drier, finally dry powder obtained. The yield was predicted to be 15˜25%.

### DETAILED DESCRIPTION OF THE INVENTION

This invention provides a method of treating viral infections with an herbal composition comprising: extracting an active ingredient from herbs of at least one species selected from genus Isatis and the genus Baphicacanthus; formulating the extract into a pharmaceutically acceptable formulation; and administering said formulation to a mammal suffering viral infection.

In an embodiment, the species is selected from the genus Isatis including Isatis tinctoria L and Isatis indigotica Fort. In another embodiment, the species is selected from the genus Baphicacanthus including Baphicacanthus cusia Bremek.

In a separate embodiment, the viral infection includes, but not limited to, the influenza virus infection, the hepatitis virus infection and human immunodeficiency virus. Other viral infections such as the enterovirus infection is applicable.

In an embodiment of the above method, it is characterized by the extraction being performed in the presence of an organic solvent. Said organic solvent includes but is not limited to ethanol, ether, or acetone.

In a different embodiment, it is characterized by the extraction being performed in the presence of water.

In another embodiment, the formulation is in the form of a powder, syrup, tea, tincture, injection, topical solution, capsule, pill, granule, tablet, nebula, suppository or microcapsule. Other pharmaceutically suitable carriers may be used.

In an embodiment above, the formulation is administered in a dosage range of 1 to 50,000 milligrams per day. In another embodiment, the range is 1 to 10,000 milligrams per day. In a still another embodiment, the range is 10 to 10,000. In a further embodiment, the range is 10 to 1,000.

In another embodiment, the extraction step is performed by: obtaining a solid combination of Isatis and Baphicacanthus; pulverizing said solid combination; extracting said solid combination with 40%-90% of an alcohol under reflex; evaporating said alcohol and extract combination to produce a liquid; adding water and a macromolecule precipitating agent to said liquid; and refining said liquid mixture through a rosin chromatographic column.

In an embodiment, the refining is performed by eluting the rosin column with distilled water; evaporating the distilled water from the eluent to produce a first extract; eluting the rosin column with 70%-98% alcohol to produce a second extract; and combining the first and second extracts.

From an above embodiment, the extraction step is performed by pulverizing a solid combination of Isatis and Baphicacanthus; boiling the solid at least two times with water, filtering the boiled water to produce a filtrate; adding a macromolecule precipitating agent to the filtrate; filtering the filtrate a second time; and placing the filtrate under vacuum to evaporate the solvent.

This invention provides a method of producing an herbal extract, wherein the extraction step is performed by: obtaining a solid combination of Isatis and Baphicacanthus; pulverizing said solid combination; extracting said solid combination with 40%-90% alcohol under reflex; evaporating the alcohol and extract combination to produce a liquid; adding water and a macromolecule precipitating agent to the liquid; and further refining the liquid mixture through a rosin chromatographic column.

From another embodiment, the refining step is performed by sequentially eluting the rosin column with distilled water; evaporating the distilled water from the eluent to produce a first extract; eluting the rosin column with 70%-98% ethanol to produce a second extract; and combining the first and second extracts.

This invention provides a method of producing an herbal extract, wherein the extraction step is performed by pulverizing a solid combination of Isatis and Baphicacanthus; boiling said combination at least two times with water, filtering said water to produce a filtrate; adding a macromolecule precipitating agent to the filtrate; filtering the filtrate a second time; and placing the filtrate under vacuum to evaporate the solvent.

An herb extract product is produced by above methods. This invention provides a pharmaceutical composition comprising an effective amount of an extract of at least one species selected from genus Isatis and the genus Baphicacanthus and a pharmaceutically acceptable formulation.

This invention provides a pharmaceutical composition comprising an effective amount of an extract of at least one species selected from genus Isatis and the genus Baphicacanthus in inhibiting viral replication in infected cells or rendering viruses noninfectious and a pharmaceutically acceptable formulation.

In a separate embodiment, the viral infection includes but is not limited to influenza virus infections, the hepatitis virus infections or the human immunodeficiency virus infections.

In an embodiment of the above, the viral infection includes infection with the hepatitis virus.

In a further embodiment, the viral infection includes infection with the human immunodeficiency virus.

A method for producing an extract from Isatis comprising steps of: a. Decoding certain amounts of Isatis species in water for an appropriate time; b. Separate the aqueous phase from the residue; c. Adding appropriate flocculant solution to the solution; d. Seperating the clear solution and precipitate; and e. Vacuum dry the separated clear solution to produce an Isatis extract.

In an embodiment, the flocculant is a chitosan. This invention also provides an extract produced from the above method. This invention provides a composition that comprises the above extracts. Finally, this invention also provides anti-viral compositions comprising of the above extracts.

First Series of Experiments

Herbs from genus Isatis belong to the mustard family and their origins can be traced back over 2000 years. In some parts of the world the plant has been cultivated for use as a source of blue dye. Isatis tinctoria, for example, produces large quantities of seed containing allelopathic compounds and has the ability to reproduce vegetatively. The root system of I. tinctoria consists of a large taproot which penetrate deep into the soil (up to 1.5 m). The leaves of I. tinctoria are of two types; basal and cauline. Basal rosette leaves are petiolate, 3.5 to 15 cm long and 0.8 to 4 cm wide, oblanceolate to elliptic. The basal rosette ranges in diameter from 3.5 and 18 cm. The cauline leaves are sessile, alternately arranged, and lanceolate. The leaves are usually blue-green in color with a cream colored mid-rib and are covered with fine hair. Approximately 20 large woody purple stalks are produced following bolting but typically fewer than seven grow to maturity. These stalks grow to 1 meter in height and are glabrous but may have a few long simple hairs at the base.

Isatis indigotica Fort

The inflorescence of I. tinctoria is an umbrella-shaped corymb with individual flowers growing to about 3 mm long and are bright yellow in color. Flowering occurs in late spring with exact timing of flowering dependent upon elevation. Hundreds of silicles (pods), each containing one seed, are produced anywhere from 4 to 6 weeks after flowering begins. The seeds of I. tinctoria are yellow and 3 to 3.5 mm in length and have a pedicel which acts as a hook, allowing transport in animal fur, on clothing, or in machinery and tires. They also have flattened wings which may assist in both wind and water dispersal.

Herbs for the genus Isatis have been known to possess medicinal properties and have been used for centuries, especially in China. According to the present invention, an improved method of extracting the active ingredients has been developed that allows for greater efficacy of the Isatis extract in the treatment and prevention of viral infections. Herbs from the genus Baphicacanthus have elliptical leaves with a shallowly serrate margin and commonly range between 5 and 12 centimeters in length. Similar to herbs from the genus Isatis, Baphicacanthus has long been used in China for the treatment of many diseases including cancer.

In practice, the present invention uses a single extraction process for concentrating the active ingredients of the Isatis and Baphicacanthus plants. This process may be carried out through the ref lux of approximately 2 kilograms of herbs from the genus Isatis and/or Baphicacanthus with between 40% and 90% ethanol for an appropriate time, for example between 2 and 4 hours. More particularly, 1900 grams of a solid combination of Isatis and Baphicacanthus are obtained and pulverized or shattered. The solid is then extracted with 40%-90% ethanol under reflex, preferably with 50%-85% ethanol. The resulting solution placed under vacuum for a time sufficient to evaporate the solvent, preferably through the use of a rotary evaporator, to produce a syrup-like liquid. Distilled water and a macromolecule precipitating agent are added to the syrup The extract is then filtered, and further refined through a rosin chromatographic column. The rosin column is eluted with distilled water until the eluent is nearly achromatic. The solvent is evaporated from the eluent, resulting in extract A (350 grams). The rosin column is then eluted with 70%-98% ethanol until the eluent is almost achromatic. The ethanol solvent is evaporated from the second eluent, resulting in extract B (23.5 grams). Extracts A (20-80 wt.%) and B (80-20 wt. %) are combined to produce extract C.

Smaller amounts of extract can be obtained by pulverizing or shattering 100 grams of a solid combination of Isatis and Baphicacanthus. The solid is then boiled 3 times with water, and filtered. A macromolecule precipitating agent is added to the filtrate. The resulting solution is filtered again, and placed under vacuum for a time sufficient to evaporate the solvent, producing a dry residue (Extract D). Such an extraction may then be formulated into a variety of administrable compositions such as powders, syrups, teas, tinctures, injections, topical solutions, capsules, pills, granules, tablets, nebulas, suppository and microcapsule formulations. The formulations may also be combined with excipients, binders and adjuvants commonly used in the formulation of herbal and nutritional supplements.

Although the present invention has been described in detail with particular reference to preferred embodiments thereof, it should be understood that the invention is capable of other different embodiments, and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only, and do not in any way limit the invention, which is defined only by the claims.

Second Series of Experiments

Extraction Process for the Sample HR3

A great deal of Chinese patent drugs derive from Banlangen, which is come from the root of Isatis indigotica Fort. It is used to relieve internal heat, detoxity and cool the blood. In clinical practice it is proved to be anti-virus, anti-endotoxin and anti-bacterial effective. The purpose of this study is to find a useful extracting process, through which the extructum from a good quality of raw herbs is effective on anti-virus. Based on the possibility of applying in industrial area, the process should be simple and the needed unit equipments should be available. The most important factor is that the final extructum should be efficiency enough for clinical practice.

According to the literature data and our analysis, the effective component in the Isatis indigotica Fort is soluble in water. For a comparative full-scale searching for the efficiency component, 7 different extracting processes were designed; and thus 7 samples are provided to professor Chen (Beijing) for anti-virus-efficiency-screening. Experiments results show the satisfactory effect of the sample HR3.

Discussion

The technology of ethanol subsiding is usually used to refining Chinese medicine preparation based on the different solubility of certain effective part of the TCM in water and ethanol. But there are some shortcuts about this technology which are described as follow:

The concentration of the solute in the water extracting solution or the target component decreased greatly during the ethanol subsiding process. Thus it's difficult to maintain the quality of the preparation. The less soluble material loss completely. Higher cost. Special equipment must be used for recovering of the ethanol and the wastage of ethanol will be about 20%.

The stability of the quality of the product is bad. The removing of hydrophilic component makes the liquid preparation easily subside and adhesive to the wall of the container. Long production time makes against the improving the economic benefits.

A great deal of research report concerning new separation and refine technology can be found in the literature, such as super-filtrate using a cosmic membrane, flocculate subside etc. The flocculate subside has many benefits in the refining of water extracting solution as following:

A little flocculent consumption and simple equipment required. Short production time. The flocculate unit takes 3˜6 hours, about half producing time reserved. Good quality of the product can be achieved. The percentage of the effective component can be improved in order to confirm the curative effect. Stability of the liquid preparation is good. No sedimentation arises.

Flocculating process is to use flocculating agent to refine traditional Chinese medicine. The flocculating agent, chitosan, is added into the water extracting solution; Colloid pellets are got rid of in a absorbing matter, such as protein, mucilage. Then filtrate to refine it. Its principle is: water extracting solution of traditional Chinese medicine has many components polymer, such as mucus, protein, starch, etc. It is unstable system and has high surface energy. When chitosan is added, big pellets are got rid of by the function of absorbing bridge and electric neutralization.

The root of Isatis indigotica Fort. is used as Banlangen, which contains a great deal of dissolvable starch with hot water. The ethanol subsiding technology is usually used to refining Chinese medicine preparation. In this study the flocculation technology was applied in HR3 preparation. The effect of sample HR3 anti-virus is convinced by the curation experiment, performed by Prof. Chen Hongshan.

### REFERENCES OF EXTRACTION PROCESS FOR RADIX ISATIDIS

[1]. Zhang Runzhen, Zhang Yuwen. “Research development on Radix Isatidis”, 2000, 31(6), 474

[2]. Liu Sheng, Chen Wansheng, Qiao Chuanzhuo, Zhen Shuiqing, Zeng Ming, Zhang Hanming, Song Zhaojun. “Antiviral Action of Radix Isatidis and Folium Isatidis from Different Germplasm Against Influenza A Virus”, Academic Journal of the Second Military Medicine University, 2000, 21(3), 204

[3]. Wang Lixia, He Yan, Chen Yonggan, Wan Li. “Research Development on the Chemical Compounds of Radix Isatidis and”, Academic Journal of Medicine School of Kai Feng, 1999, 18(3), 52

[4]. Deng Huimin, Su Biru, Chen Yanyang. “Application of Cross-experiment design in optimization of extracting process for Radix Isatidis and Rhizoma et Radix Bahicacanthis Cuisiae”, Research & Information on Traditional Chinese Medicine, 1999, 18(3), 52

[5]. Wang Li, Lu Jianjiang, Gu Chenzhi, Cheng Yuhuai. “Application of Micro-wave technology in Radix Isatidis Polysaccharide extraction and measurement”, Chinese Traditional and Herbal Drugs, 2001, 24(3), 180

[6] Zhang Tong, Xu Lianying, Cai Zhenzhen. “Effects of clarificant chitosan on contents of Zinc, Manganese, Calcium and heavy metal lead in water-extraction liquid of Chinese Herbal Medicines”, Chinese Traditional Patent Medicine, 2001,23(4), 243

[7]. Liu Sizhen, Zhu Xixian, Shao Yuqin, Ma Tianbo. “Screening of effective part against Influenza in Radix Isatidis”, Chinese Traditional and Herbal Drugs, 1999, 30(9), 650

Third Series of Experiments

In Vitro and In Vivo Anti-viral Activities of Chinese Herb Isatis indigotica Fort. On Influenza Viruses by Institute of Medical Biotechnology, Chinese Academy of Medical Sciences, Beijing, China

Isatis indigotica Fort is a kind of widely distributed Chinese herb and used as folk remedy for flu in China, its common name is “Ban Lan Gen”. Its pharmacological function has been recorded in Chinese traditional medicine book as “clean heat and resolve toxicity”. Ban Lan Gen preparations: oral liquid, pills, and granules have been marketed and used in clinics popularly in China. Some papers reported BLG was effective on prevention and treatment of influenza (1,2,3,4). 999 Pharmaceutical Company in Shengzhen, Guangdong, China intended to develop it as anti-influenza herb medicine and designed a project to study its anti-influenza active components and to confirm its antiviral activities.

Isatis indigotica Fort., is belong to Cruciferae, its water decoctions have been reported to inhibit influenza virus A/86-1 in infected chicken embryos(5,6,7). This report showed that 3 extracts of the herb inhibited influenza viruses type A and B in MDCK cell cultures(8).

In this study, Ban Lan Gen (BLG) herbs were collected, identified and extracted by National Engineering Research Center for Traditional Chinese Medicine in Shanghai, China, the BLG extracted samples were supplied for in vitro and in vivo anti influenza viral studies.

The anti-influenza drugs Ribavirin(9,10) and Amantadine(11,12) were used as positive control in in vitro and in vivo tests respectively.

Materials and Methods

1. Materials:

1.1. Ban Lan Gen(BLG) extracts: Water extracts: HR1, HR2, HR3, HR4, alcohol and water extracts MW2, AT1 and AA2., all were lyophilized to be dry powders, stored at room temperature. Prepared by National Engineering Research Center for Traditional Chinese Medicine in Shanghai, China.

1.2 Viruses: Influenza viruses strains: type A/90-15strain, type B/97-13 and type A/FM1-mouse lung adapted strain.

Inoculated into chicken embryos and infected chicken embryo allantoic fluids collected, stored in 80° C. in refrigerator.

1.3 Cell cultures: MDCK cells cultures in 96 holes plates, incubated in 5% CO₂ at 37° C. in incubator.

1.4 Mice: KM species, SPF 3rd grade, female, body weight: 14-16 grams. Purchased from Center for Laboratory Animals, National Institute of Drug and Biological Standardization.

1.5 Anti-influenza drugs: Ribavirin(1-β-D-ribafuranosyl-1,2,4-triazole-3-carboxamide) and Amantadine. Purchased from Yukang Pharmaceutical Company in Zhejiang province.

2. Methods:

2.1 Preparation of viruses stocks.

For cell culture studies: Influenza virus type A (H₃N₂) strain Jifeng A/90-15, Influenza virus type B strain Jifeng B/97-13.infected chicken embryos to allantoic sacs, after incubation allantoic fluids were collected. Influenza viruses A and B were Inoculated into MDCK cell cultures to titrate TCID₅₀ for virus cell infectivities.

For mice studies: Influenza virus type A (H₁N₁) FM₁ mouse lung adapted strain infected mouse lungs were grounded and the suspension inoculated into chicken embryos, allantoic fluids were collected and inoculated to mouse nostrils under ether anesthesia to titrate LD₅₀ for virus infectivity in mice.

2.2. Preparation of samples:

For MDCK cell culture: The extracts HR1, HR2, HR3, HR4 MW2 and AT1 were dissolved in culture medium to made 10 mg/ml solutions, AA2 dissolved in DMSO and diluted with culture medium to made 0.2 mg/ml of 30% DMSO suspension.

For mice: The extracts HR1, HR2, HR3 HR4, MW2 and AT1 were prepared with 0.3% oxy-methyl-cellulose to make homogenetic suspensions. The extract AA2 was dissolved in DMSO and then diluted with water to make 30% DMSO homogenetic suspension.

2.3. In vitro: anti-influenza virus assays in cell cultures:

2.3.1.

Drug toxicity in Cell culture: 24 hrs MDCK cell culture in 96 holes plates, drug samples were added with 6 concentrations of 2 fold dilutions, 3 holes per concentrations. Cultured at 37° C. in CO2 incubator for 72 hours. Recorded the degrees of cytopathic effects (CPE): The grade criteria were: “0”: no CPE:, “0.5”: 5-10% CPE:, “1”: 10-25% CPE:, “2”:25-50% CPE:, “3”: 50-75% CPE,. “4”: 75-100% CPE: Calculated the 50% toxic concentration (TC₅₀) and maximum non-toxic concentration (TC₀)

2.3.2.

Drug inhibition assays on influenza viruses type A and B in infected MDCK cell cultures:

Early treatment assay: Cell cultures infected with 10⁻³-10⁻⁴ concentration of Influenza viruses type A and B, 2 hours later poured off virus fluids and washed with medium. Add 2 fold dilutions of TC₀ concentrations of drug samples to infected cell plates, 3 holes per concentration. Incubated and recorded CPE after 72 hours. Set virus control, solvent control and different concentration of Ribavirin as positive drug controls.

Calculated inhibition % of virus CPE of drug samples in comparison with solvent control. Inhibition % >50% was considered effective.

Preventive assay: Cell culture plate pre-treated with different concentrations of sample solutions for 24 hours, washed out the drugs, then infected with viruses, incubated, washed, observed and calculated the inhibition % as above.

2.2.4.

In vivo: anti-influenza viruses studies in mice.

2.2.4.1.

Acute oral toxicity in mice: Mice given orally with different doses of BLG samples, Observed and recorded the toxic reaction and mortality for 7 days.

2.2.4.2.

Inhibition of lung lesions in influenza virus infected mice. Mice infected intranasally with Influenza virus type A FM₁ mouse adapted strain, 2 hours later, BLG samples were given to groups of infected mice with 2-3 different non-toxic doses by oral route, bid for 4 days. Set up virus control, vehicle control and positive drug: amantadine control. On the 4^{th} day, killed all the mice, removed the whole lungs. Record the lung lesions in each group. Calculated the inhibition % of lung lesions in mice of drug treated groups in comparison with that in solvent group. Inhibition %>50% was considered effective.

2.2.4.3.

Protection of mortality and prolongation of survival days by BLG samples in influenza virus infected mice.

Mice infected and given with BLG samples. But the treatments were prolonged to 14 days, the mortality and death dates were recorded. Set controls as above. Calculated the % of protection of average mortality rate and prolongation of average survival days of BLG samples in comparison with vehicle control. Effective % was >50%.

Results

1. In vitro Studies:

1.1. Toxicity of 7 Kinds of BLG Samples in MDCK Cell Cultures.

7 BLG samples were firstly tested in MDCK cell cultures for cellular toxicity to find the out the 50% toxic concentrations (TC₅₀) and maximum tolerable concentrations (TC₀). The results showed in Table 1.

1.2. Inhibition of influenza viruses CPE of 7 kinds of BLG samples in MDCK cell cultures.

MDCK cell cultures infected with influenza viruses A/90-15 and B/97-13, 2 hours later added 6 concentrations of 2-fold dilutions of these 7 samples started from maximum tolerable concentration.

In comparison with the virus infected vehicle control, HR3, HR4 and MW2 were found to inhibit virus CPE, 50% inhibition concentrations (IC50) and selective indexes (SI) of HR3 were seen in Table 1. The results were summarized as follows:

HR3: For Influenza virus A/90-15: IC₅₀: 0.94±0.11, SI: 4.93. For Influenza virus B/97-13: IC₅₀: 1.13±0.13, SI: 4.10.

HR4: For Influenza virus A/90-15: IC₅₀: >1.21±0.25, SI: <3.94.

For Influenza virus B/97-13: IC₅₀: >1.35±0.25, SI: <3.90.

MW2: For Influenza virus A/90-15: IC₅₀: 0.20±0.04, SI: 3.98.

For Influenza virus B/97-13: IC₅₀: >020±0.04, SI: <3.90.

HR4 IC50 was near to its maximum tolerable concentration. All the other 4 samples showed no inhibition on their maximum tolerable concentrations.

Anti-influenza Virus Drug Ribavirin:

For Influenza virus A/90-15: IC₅₀: 0.07±0.04, SI: >14.29.

For Influenza virus B/97-13: IC₅₀: 0.06±0.03, SI: >16.67.

HR3 was the best extract with higher SI, MW2 was the second. All the other 5 extracts showed weak or no anti-influenza virus activity.

TABLE 1
Cellular toxicity and inhibition of CPE of
influenza viruses infected MDCK cell cultures

| | | | | | Influenza virus | |
|---|---|---|---|---|---|---|
| | | | | | B/97-13 | |
| | TC₅₀ | TC₀ | Influenza virus A/90-15 | | IC₅₀ | |
| Sample | mg/ml | mg/ml | IC₅₀ mg/ml | SI | mg/ml | SI |
| HR1 | 6.19 ± 1.68 | 1.25 ± 0 | *>1.5 | | | |
| HR2 | 4.48 ± 0.30 | 1.25 ± 0 | *>1.5 | | | |
| HR3 | 4.63 ± 0.52 | 1.25 ± 0 | 0.94 ± 0.11 | 4.93 | 1.13 ± 0.13 | 4.10 |
| HR4 | 4.77 ± 0.71 | 1.25 ± 0 | >1.21 ± 0.25 | <3.94 | >1.35 ± 0.25 | <3.53 |
| MW2 | 0.78 ± 0.39 | 0.28 ± 0.04 | 0.20 ± 0.04 | 3.98 | >0.20 ± 0.04 | <3.90 |
| AT1 | 1.43 ± 0.60 | 0.41 ± 0.13 | *>0.41 | | 0.41 ± 0.08 | |
| AA2 | 0.05 | 0.025 | *>0.025 | | *>0.025 | |
| Ribavirin | >1.00 | >1.0 | 0.07 ± 0.04 | >14.29 | 0.06 ± 0.02 | >16.67 |

*Maximum tolerable concentration TC₀ no effect.

1.3. Repeated Anti-influenza virus activities of HR3, HR4 and MW2 with new batches of samples.

In order to confirm the anti-influenza viruses activities, 3 batches of HR3 and 2 batch of MW2 were tested again in MDCK cell cultures infected with different doses of viruses. The results were shown in Table 2. and 3.

The results showed the regular rule of antiviral studies; the virus-infecting concentrations are reversibly related to the anti-viral activities. Decreased the virus-infecting dosage, decreased IC50 value and increased SI value.

With 10-20 TCID₅₀ influenza virus infection,

HR3: Batch 1 and 2 showed higher anti-viral activity on type A, lower activity on type B.

For A/90-15: IC₅₀: 0.69±0 mg/ml, SI: 6.93

For B/97-13: IC₅₀: 1.19±0 mg/ml, SI: 3.89 Batch 3 showed lower anti-viral activities.

MW2: Batch 1 and 2 showed same anti-viral activities on influenza viruses type A and B

For both A/90-15 and B/97-13: IC₅₀: 0.18 mg/ml, SI: 4.43

TABLE 2
Inhibition of 3 batches of HR3 on influenza viruses
A and B in MDCK cell cultures.

| | | Influenza virus A/90-15/Infecting conc.(TCID₅₀) | | | | | |
|---|---|---|---|---|---|---|---|
| | | 10⁻⁴(177.8-213.8) | | 10⁻⁵(17.78-21.4) | | 10⁻⁶(1.78) | |
| Sample | Batch | IC₅₀(mg/ml) | SI | IC₅₀(mg/ml) | SI | IC₅₀(mg/ml) | SI |
| HR3 | 1 | 1.19 ± 0 | 3.89 | 0.69 ± 0 | 6.93 | 0.69 | 6.93 |
| | 2 | 1.125 ± 0.09 | 4.11 | 0.69 ± 0 | 6.93 | 0.69 | 6.93 |
| | 3 | *1.50 | | 1.03 ± 0.22 | 4.48 | 1.13 | 4.07 |
| | | Influenza virus B/97-13/Infecting conc.(TCID50) | | | | | |
| | | 10⁻³(16.98) | | 10^{-3.3-4}(8.49-10) | | 10⁻⁵(1) | |
| Sample | Batch | IC₅₀(mg/ml) | SI | IC₅₀(mg/ml) | SI | IC₅₀mg/ml | SI |
| HR3 | 1 | *1.5 | | 1.19 ± 0 | 3.89 | 0.88 | 5.26 |
| | 2 | *1.5 | | 1.19 ± 0 | 3.89 | 0.88 | 5.26 |
| | 3 | *1.5 | | 1.19 ± 0 | 3.89 | 1.19 | 3.89 |

*Maximum tolerable concentration TC₀ no effect.

TABLE 3
Inhibition of 2 batches of MW2 on influenza viruses
A and B in MDCK cell cultures.

| | | Influenza virus A/90-15/Infecting conc.(TCID₅₀) | | | |
|---|---|---|---|---|---|
| | | 10⁻⁴(213.8) | | 10⁻⁵(21.38) | |
| Sample | Batch | IC50(mg/ml) | SI | IC50(mg/ml) | SI |
| MW2 | 1 | 0.20 | 3.94 | 0.18 | 4.43 |
| | 2 | 0.20 | 3.94 | 0.18 | 4.43 |
| | | Influenza virus B/97-13/Infecting conc.(TCID50) | | | |
| | | 10⁻³(16.98) | | 10^{-3.3}(8.49) | |
| Sample | Batch | IC50(mg/ml) | SI | IC50(mg/ml) | SI |
| MW2 | 1 | *0.25 | | 0.18 | 4.43 |
| | 2 | *0.25 | | 0.18 | 4.43 |

*Maximum tolerable concentration TC₀ no effect.

2. In vivo Studies:

2.1. Acute Toxicity of 7 Kinds of BLG Extracts in Mice by Oral Administration.

Different soages of the 7 BLG samples suspensions were given to groups of mice by oral administration, once, observed toxic reactions and death of mice on 7th day. The results were as follows:

| | |
|---|---|
| HR1, HR2, HR3, HR4, MW2: | 25 g/kg no toxicity. |
| AT1: | 25 g/kg 1/3 died. |
| AA2: | 10 g/kg no toxicity. |

2.2. Inhibition of 7 BLG Extracts on Influenza Virus Lung Lesions in Infected Mice.

Mice infeected by intranasal route with Influenza virus A/FM1 mouse adapted strain, all 7 BLG extracts were given to infected mice by oral with 2-3 different non-toxic doses, bid for 4 days. Mice were sacrafised and the lungs were token to score the lesions.

The average lesion scores of the treated mice were compared with that of the vehicle control and calculated the inhibition %, The inhibition above 50% was considered to be effective.

Only HR3, HR4 and MW2 inhibited mice lung lesions for more than or about 50%. The results were shown in Table 4.

The inhibition % of HR3, HR4 and MW2 on influenza lung lesions were summerazed as follows:

| | | | |
|---|---|---|---|
| HR3: | 10 g/kg bid × 4: | Inhibition % | 55.35% |
| | 5 | | 50.00% |
| | 2.5 | | 28.57% |
| HR4: | 10 | | 78.575% |
| | 5 | | 71.43% |
| | 1.5 | | 42.86% |
| MW2 | 5 | | 49.09% |
| | 2.5 | | 34.55% |
| | 1.25 | | 56.36% |
| Amantadine | 0.10 | | 100.00% |
| | 0.05 | | 85.71% |
| | 0.025 | | 78.57% |

All the tolerable dosages of the other 5 BLG extracts showed no or low inhibition % on influenza lung lesions and are considered to be ineffective.

TABLE 4
The early therapeutic effects of 7 kinds of BLG
samples on lung lesions in type A/FM1/mouse adapted
influenza virus infected mice.

| | Sample dose | Mice | | Average lung | Inhibition 5 of |
|---|---|---|---|---|---|
| Sample | g/kg | no. | Death rate | lesion score | lung lesions |
| HR1 | 10 | 5 | 0/5 | 2.4 ± 0.55 | 14.29 |
| | 5 | 5 | 0/5 | 2.2 ± 1.30 | 21.43 |
| | 2.5 | 5 | 0/5 | 2.0 ± 1.0 | 28.57 |
| HR2 | 10 | 5 | 0/5 | 2.6 ± 0.55 | 7.14 |
| | 5 | 5 | 0/5 | 1.8 ± 0.45 | 35.71 |
| | 2.5 | 5 | 0/5 | 1.6 ± 0.45 | 42.86 |
| HR3-1 | 10 | 5 | 1/5 | 1.25 ± 0.5 | 55.36 |
| | 5 | 5 | 0/5 | 1.4 ± 0.55 | 50.00 |
| | 2.5 | 5 | 0/5 | 2.0 ± 0.71 | 28.57 |
| HR4-1 | 10 | 5 | 0/5 | 0.6 ± 0.55 | 78.57 |
| | 5 | 5 | 0/5 | 0.8 ± 1.10 | 71.43 |
| | 2.5 | 5 | 0/5 | 1.6 ± 0.55 | 42.86 |
| MW2-1 | 5 | 5 | 0/5 | 1.4 ± 0.55 | 49.09 |
| | 2.5 | 5 | 0/5 | 1.8 ± 0.84 | 34.55 |
| | 1.25 | 5 | 0/5 | 1.2 ± 0.045 | 56.36 |
| AT1 | 5 | 5 | 1/5 | 2.75 ± 0.5 | 0 |
| | 2.5 | 5 | 0/5 | 2.0 ± 0 | 27.27 |
| | 1.25 | 5 | 0/5 | 3.0 ± 1.0 | 9.09 |
| 0.3% | | 4 | 0/4 | 2.75 ± 1.26 | |
| OMC | | | | | |
| Control | | | | | |
| AA2 | 5 | 5 | 2/5 | 2.0 ± 1.0 | 0 |
| | 2.5 | 5 | 0/5 | 1.4 ± 0.55 | 30 |
| | 1.25 | 5 | 0/5 | 2.2 ± 0.84 | 10 |
| 30% | | 4 | 0/4 | 2 ± 0.82 | 0 |
| DMSO | | | | | |
| Control | | | | | |
| Aman- | 0.10 | 5 | 0/5 | 0 ± 0 | 100 |
| tadine | 0.05 | 5 | 0/5 | 0.4 ± 0.55 | 85.71 |
| | 0.025 | 5 | 0/5 | 0.6 ± 0.55 | 78.57 |

2.3. Confirmation of anti-influenza viral activities of MR3 and MW2 in mice. In order to confirm the in vivo anti-influenza virus activities of HR3 and MW2 in mice, 2 kinds of experiments were done.

2.3.1.

Inhibition of influenza mouse lung lesions in mice: to repeat the previous experiment for evaluation the efficasy.

2.3.2.

Protection of death in influenza virus pneumonia mice, to calculate % of protection of death rate and prolongation of life span.

The results were summerized as follows:.

For HR3 (Table 5).

Inhibition % of Lung Lesions:

10 g/kg: Exp.1: 55.36%, Exp. 2: 30.63%;

average: 43.00±17.49%.

5 g/kg: Exp.1: 50.00%, Exp. 2: 60.86%;

average: 55.43±7.68%.

2.5 g/kg: Exp.1: 28.57%.

Protection of death and prolongation of life span:

10 g/kg: Exp.1: 50.00% and 51.85%,

5 g/kg: Exp.1: 50.00% and 55.56%, Exp. 2: 50.00% and 55.83%

average: 50.00±0% and 55.70±0.19%.

For MW2(Table 6).

Inhibition % of Lung Lesions:

5 g/kg: Exp.1: 49.09%, Exp. 2: 51.35%;

average: 50.23±1.61%.

2.5 g/kg: Exp.1: 34.55%, Exp. 2: 36.58%;

average: 35.57±1.44%.

1.25 g/kg: Exp.1: 56.36%.

Protection of death and prolongation of life span:

10 g/kg: Exp.1: 0, and 12.96%,

5 g/kg: Exp.1: 37.50% and 40.74%, Exp. 2: 12.50% and 13.46%

average: 25.00±17.68% and 27.10±19.29%.

2.5 g/kg: Exp. 2: 0, −1.92%.

For Amantadine: (Table 5,6)

Inhibition % of Lung Lesions:

0.1 g/kg: Exp.1: 100.00%, Exp. 2:74.54%; Exp. 4. 95.14%,

average: 89.89±13.52%.

0.05 g/kg: Exp.1: 85.71%,

0.025 g/kg: Exp.1: 78.57%.

Protection of death and prolongation of life span:

0.1 g/kg: Exp.1: 85.71% and 76.67%, Exp.2. 75.00% and 72.75%, Exp. 3. 37.50% and 63.46%.

average: 0.1 g/kg: Protection of death: 66.07±25.32%,

Prolongation of life span: 70.96±6.78%.

TABLE 5
Inhibition of HR3 on lung lesions, protection of
mortality and prolongation of life span in mouse adapted
influenza virus A/FM1 infected mice.

| | | | Sample | Inhibition of lung lesions | | |
|---|---|---|---|---|---|---|
| | Influenza | | Dosage | Death | Av. Lung | Inhibition |
| Exp. | virus A/FM1 | Sample | g/kg | rate | lesion score | % |
| 1 | 10^{-3.48} | HR3-1 | 10 | 0/5 | 1.25 ± 0.50 | 55.36 |
| | (41.60LD₅₀) | | 5 | 0/5 | 1.40 ± 0.55 | 50.00 |
| | | | 2.5 | 0/5 | 2.0 ± 0.71 | 28.57 |
| | | Vehicle | 0 | 0/5 | 2.80 ± 0.84 | |
| | | control | | | | |
| | | Amantadine | 0.1 | 0/5 | 0 | 100 |
| 2 | 10^{-3,48} | HR3-2 | 10 | 0/8 | 1.88 ± 0.84 | 30.63 |
| | (41.60LD₅₀) | | 5 | 0/8 | 1.06 ± 1.02 | 60.86 |
| | | Vehicle | 0 | 1/8 | 2.71 ± 0.49 | |
| | | control | | | | |
| | | Amantadine | 0.1 | 0/8 | 0.69 ± 0.26 | 74.54 |
| | | | | Mortality protection | | |
| | Influenza | | Sample | | Protection | Prolongationo |
| | virus A/FM1 | | dosage | Death | of death | of life span |
| Exp. | Virus dosage | Sample | g/kg | rate | % | % |
| 1 | 10⁻⁴ | HR3-1 | 10 | 4/8 | 50.00 | 51.85 |
| | (12.5LD₅₀) | Vehicle | 5 | 4/8 | 50.00 | 55.56 |
| | | control | 0 | 8/8/ | | |
| | | Amantadine | 0.1 | 1/8 | 85.71 | 76.67 |
| 2 | 10⁻⁴ | HR3-2 | 10 | | | |
| | (11.22 LD₅₀) | Vehicle | 5 | 4/8 | 50.00 | 55.83 |
| | | control | 0 | 8/8 | | |
| | | Amantadine | 0.1 | 2/8 | 75.00 | 72.75 |

TABLE 6
Inhibition of MW2 on lung lesions, protection of
mortality and prolongation of life span of mouse adapted
influenza virus A/FM1 infected mice

| | | | Sample | Inhibition of lung lesion | | |
|---|---|---|---|---|---|---|
| | Influenza virus | | Dosage | Death | Protection of | Prolongation |
| Exp. | A/FM1(conc.) | Sample | g/kg | rate | death % | of life span % |
| 1 | 10^{-3 48} | MW2 | 5 | 0/5 | 1.40 ± 0.55 | 49.09 |
| | (41.60LD₅₀) | | 2.5 | 0/5 | 1.80 ± 0.84 | 34.55 |
| | | | 1.25 | 0/5 | 1.20 ± 0.05 | 56.36 |
| | | Vehicle control | 0 | 0/5 | 2.75 ± 1.26 | |
| | | Amantadine | 0.1 | 0/5 | 0 | 100 |
| 2 | 10^{-3 48} | MW2 | 5 | 0/8 | 1.25 ± 1.4 | 51.36 |
| | (41.60LD₅₀) | | 2.5 | 0/8 | 1.63 ± 0.74 | 36.58 |
| | | Vehicle control | 0 | 1/8 | 2.57 ± 0.79 | |
| | | Amantadine | 0.1 | 0/8 | 0.13 ± 0.35 | 95.14 |
| | Influenza virus | | Sample | Mortality protection | | |
| | A/FM1 | | dosage | | | Prolongation |
| Exp. | Infecting Conc. | Sample | g/kg | Death rate | Protection % | of life span % |
| 1 | 10⁻⁴ | MW2-2 | 10 | 8/8 | 0.00 | −12.96 |
| | | | 5 | 5/8 | 37.50 | 40.74 |
| | | Vehicle control | 0 | 8/8/ | | |
| | | Amantadine | 0.1 | 1/8 | 85.71 | 76.67 |
| 2 | 10⁻⁴ | MW2-2 | 5 | 7/8 | 12.5 | 13.46 |
| | | | 2.5 | 8/8 | 0.00 | −1.92 |
| | | Vehicle control | 0 | 8/8 | | |
| | | Amantadine | 0.1 | 5/8 | 37.5 | 63.46 |

Conclusion

Isatis indigotica Fort. is a Chinese herb, named as “Ban Lab Gen” in China and used as folk remedy for flu.

7 kinds of Ban Lan Gen (BLG) extracts: HR1, HR2, HR3, HR4, MW2, AT1 and AA2 were prepared by National Engineering Research Center for Traditional Chinese Medicine in Shanghai, China.

The MDCK cell cultures of Influenza viruses type A/90-15 and type B/97-13 were used for in vitro studies of anti-influenza virus activities of the 7 BLG extracts. 3 batches of HR3 and 2 batches of MW2 were proved to inhibit both type A and B influenza viruses when the drugs added 2 hours after infection, but not active on pretreatment for 24 hours.

For HR3:

on influenza virus A/90-15: IC₅₀ was: 0.69±0 mg/ml, SI was: 6.93.

on influenza virus B/97-13: IC₅₀ was: 1.19±0 mg/ml, SI was: 3.89.

For MW2: on influenza virus A/90-15 and

B/97-13: both IC₅₀ were: 018±0 mg/ml, SI were: 4.43.

Mice model infected intranasally with influenza virus type A/FMl mouse adapted strain, % of inhibition of lung lesions, protection of death rate and prolongation of life span of BLG samples in comparison with vehicle control were used as criteria to evaluate the anti-influenza virus activities in vivo. BLG extract samples HR3 and MW2 given orally 2 hours after infection with tolerable dosages bid for 4-14 days were proved effective.

For HR3:

5-10 g/kg were effective.

10 g/kg: Inhibition% of lung lesions (treatment for 4 days): 43.00±17.49%

Protection of death rate (treatment for 14 days): 50.00%

Prolongation of life span(treatment for 14 days): 51.85%

5 g/kg: Inhibition% of lung lesions(treatment for 4 days): 55.43≧7.68%

Protection of death rate(treatment for 14 days): 50.00±0%

Prolongation of life span(treatment for 14 days): 55.70±0.19%

For MW2:

10 g/kg was toxic and no effect, 5 g/kg inhibited lung lesions but not protection of mortality.

5 g/kg: Inhibition% of lung lesions(treatment for 4 days): 50.23±1.61%

Protection of death rate(treatment for 14 days): 25.00±17.68%

Prolongation of life span(treatment for 14 days): 27.10±19.29%

5. Known antiviral drug: Ribavirin for influenza viruses types A and B was used as positive control in MDCK cell cultures, anti-influenza type A drug: Amantadine was used as positive control in type A influenza virus infection in mice. The results revealed:

Ribavir was effective both on types A and B influenza viruses by adding to cell cultures 24 hours before or 2 hours after infection.

Amantadine 0.025-0.1 g/kg bid for 4-14 days by oral 2 hours after infection were effective.

### REFERENCES

[1]. Wentian Xie, Yiyi Xu, BLG Oral liquid preparation and effect on influenza in children. China modern Application of Medicine. 2000, 6, 28; 17 (3):251

[2]. Xuehuai Feng: Anti-influenza Experience of BLG decoction by oral administration, Xinjiang Traditional Chinese Medicine. 1998, 6, 15; 16(2), 12.

[3]. Shengrong Ding: Anti-influenza Experience of BLG Transaction,Gansu College of TCM 1992, (2), 46;

[4]. Yaoju Zhu: Experience of BLG curing Children VURTI.

Qinghai Medecine, 1996, 4, 20, 26 (4), 43-44.

[5]. Sheng Liu, Wansheng Chen, et al., Transaction of Second Military University. 2000, 3, 30; 21 (3), 204-206.

[6]. Siyuan Liu: Transaction of Jiangxi College of TCM 1999, 12, 15; 11 (4), 183,

[7]. Sixian Liu, Xixian Zhu et al., Chinese traditional Medicine, 1999, 9, 25, 30 (9), 650-651.

[8]. Hongshan Chen, Li Teng: 2001 internal material of Chinese academic of Medicine

[9]. Tisdale M, Bauer D J, The relative potencies of anti-influenza compounds, ANN. N. Y. Acad. Sci, 1977, 284, 254-263.

[10]. Shuqin Kang, Shengyong Cai, Li Teng, Hongshan Chen, intro and vitro anti-virus effect of Ribavirin, MicroBiology 1980, 20, 208-212

[11]. Shigeta S, Konno K, Yokota T, et al, Comparative activities of several nucleoside analogs against influenza A,B and C ciruses in vitro, Antimicrobial agents and chemotherapy, 1988, 32, (6), 906-911.

[12] . Davis, WL, Grunert R R, Haff R F, et al, Antiviral activity of 1-admantanamine (amantadine), Science, 1964, 144, 862-863.

[13]. Hongshan Chen, Li Teng, Amantading Hydrochloride and Rimantadine anti influenza virus effect in cell cultivation and mice (internal material.)

What is claimed:

1. A method of treating viral infection with an herbal composition comprising: extracting an active ingredient from herbs of at least one species selected from genus Isatis and the genus Baphicacanthus; formulating the extract into a pharmaceutically acceptable formulation; and administering said formulation to a mammal suffering viral infection.

2. A method according to claim 1, wherein the species selected from the genus Isatis include Isatis tinctoria L and Isatis indigotica Fort.

3. A method according to claim 1, wherein the species selected from the genus Baphicacanthus include Baphicacanthus cusia Bremek.

4. A method according to claim 1, wherein the viral infection includes infection with the influenza virus.

5. A method according to claim 1, wherein the viral infection includes infection with the hepatitis virus.

6. A method according to claim 1, wherein the viral infection includes infection with the human immunodeficiency virus.

7. A method according to claim 1, further characterized by the extraction being performed in the presence of an organic solvent.

8. A method according to claim 1, further characterized by the extraction being performed in the presence of ethanol ether or acetone.

9. A method according to claim 1, further characterized by the extraction being performed in the presence of water.

10. A method according to claim 1, wherein the formulation is in the form of a powder, syrup, tea, tincture, injection, topical solution, capsule, pill, granule, tablet, nebula, suppository or microcapsule.

11. A method according to claim 10, wherein the formulation is administered in a dosage range of 50 to 50,000 milligrams per day.

12. A method according to claim 1, wherein the extraction step is performed by:
obtaining a solid combination of Isatis and Baphicacanthus; pulverizing said solid combination; extracting said solid combination with 40%-90% of an alcohol under reflex; evaporating said alcohol and extract combination to produce a liquid; adding water and a macromolecule precipitating agent to said liquid; and refining said liquid mixture through a rosin chromatographic column.

13. A method according to claim 12, wherein said refining is performed by eluting the rosin column with distilled water; evaporating the distilled water from the eluent to produce a first extract; eluting the rosin column with 70%-98% alcohol to produce a second extract; and combining the first and second extracts.

14. A method according to claim 1, wherein the extraction step is performed by pulverizing a solid combination of Isatis and Baphicacanthus; boiling the solid at least two times with water, filtering the boiled water to produce a filtrate; adding a macromolecule precipitating agent to the filtrate; filtering the filtrate a second time; and placing the filtrate under vacuum to evaporate the solvent.

15. A method of producing an herbal extract, wherein the extraction step is performed by:
obtaining a solid combination of Isatis and Baphicacanthus; pulverizing said solid combination; extracting said solid combination with 40%-90% alcohol under reflex; evaporating the alcohol and extract combination to produce a liquid; adding water and a macromolecule precipitating agent to the liquid; and further refining the liquid mixture through a rosin chromatographic column.

16. A method according to claim 15, wherein the refining step is performed by sequentially eluting the rosin column with distilled water; evaporating the distilled water from the eluent to produce a first extract; eluting the rosin column with 70%-98% ethanol to produce a second extract; and combining the first and second extracts.

17. A method of producing an herbal extract, wherein the extraction step is performed by pulverizing a solid combination of Isatis and Baphicacanthus; boiling said combination at least two times with water, filtering said water to produce a filtrate; adding a macromolecule precipitating agent to the filtrate; filtering the filtrate a second time; and placing the filtrate under vacuum to evaporate the solvent.

18. The herb extract product by the method of claim 15-17.

19. A pharmaceutical composition comprising an effective amount of an extract of at least one species selected from genus Isatis and the genus Baphicacanthus and a pharmaceutically acceptable formulation.

20. A pharmaceutical composition comprising an effective amount of an extract of at least one species selected from genus Isatis and the genus Baphicacanthus in inhibiting viral replication in infected cells or rendering viruses noninfectious and a pharmaceutically acceptable formulation.

21. The pharmaceutical composition of claim 20, wherein the species selected from the genus Isatis include Isatis tinctoria L and Isatis indigotica Fort.

22. The pharmaceutical composition of claim 20, wherein the species selected from the genus Baphicacanthus include Baphicacanthus cusia Bremek.

23. The pharmaceutical composition of claim 20, wherein the viral infection includes infection with the influenza virus.

24. The pharmaceutical composition of claim 20, wherein the viral infection includes infection with the hepatitis virus.

25. The pharmaceutical composition of claim 20, wherein the viral infection includes infection with the human immunodeficiency virus.

26. A method for producing an extract from Isatis comprising steps of:
a. Decoding certain amounts of Isatis species in water for an appropriate time;
b. Separate the aqueous phase from the residue;
c. Adding appropriate flocculant solution to the solution;
d. Seperating the clear solution and precipitate; and
e. Vacuum dry the separated clear solution to produce an Isatis extract.

27. The method of claim 26, whereas the flocculant is a chitosan.

28. The extract produced by claim 26 or 27.

29. A composition comprising the extract of claim 28.

30. An anti-viral composition comprising effective amount of the extract of claim 28.
